(19) United States
(12) Patent Application Publication
(10) Pub. No.: US 2005/0021236 A1
(43) Pub. Date: Jan. 27, 2005

(54) Statistically identifying an increased risk for disease

(75) Inventors: Christopher Aston, Shawnee, OK (US); David Ralph, Edmond, OK (US)

Correspondence Address: FULBRIGHT & JAWORSKI L.L.P., 600 CONGRESS AVE., SUITE 2400, AUSTIN, TX, 78701, US

(73) Assignee: Oklahoma Medical Research Foundation and InterGenetics, Inc.

(21) Appl. No.: 10/778,903

(22) Filed: Feb. 13, 2004

Related U.S. Application Data
(60) Provisional application No. 60/447,600, filed on Feb. 14, 2003.

(51) Int. Cl. G06F 19/00; G01N 33/48

(52) U.S. Cl. 702/19

(57) ABSTRACT

Methods and computer readable media for statistically identifying an increased risk for disease. In one embodiment, resampling techniques are utilized to consider different genotype combinations within a resampling subset of a case/control data set. Odds-ratios and theoretical p-values are calculated for each genotype combination so that an increased risk of disease associated with a particular genotype combination may be identified. In another embodiment, different genotype combinations within a case/control data set are considered. Odds ratios are calculated for each genotype combination. Empirical p-values are calculated for the odds ratios through randomization techniques. Using the odds-ratios and/or empirical p-values, an increased risk for disease associated with a particular genotype combination may be identified.

This application claims priority to and incorporates by reference U.S. Provisional Patent Application Ser. No. 60/447,600, which was filed on Feb. 14, 2003.

### REFERENCE TO APPENDIX

This application includes a computer program listing appendix, submitted on compact disc (CD). The content of the CD is incorporated by reference in its entirety and forms a part of this specification. The content of the CD was included within the specification of U.S. Provisional Patent Application Ser. No. 60/447,600. The CD contains the following file:

| File name | File size | Creation date for CD |
|---|---|---|
| SOURCE.txt | 40 kb | Feb. 13, 2004 |

### BACKGROUND OF THE INVENTION

1. Field of the Invention

The present invention relates generally to statistical methods finding application in the life sciences. More particularly, the present invention relates to bioinformatic techniques to statistically identify an increased risk for disease, such as but not limited to, breast cancer associated with one or more particular genotype combinations or other exposure factors.

2. Background

For patients with cancer, early diagnosis and treatment are the keys to better outcomes. In 2001, there are expected to be 1.25 million persons diagnosed with cancer in the United States. Tragically, in 2001, over 550,000 people are expected to die of cancer. To a very large extent, the difference between life and death for a cancer patient is determined by the stage of the cancer when the disease is first detected and treated. For those patients whose tumors are detected when they are relatively small and confined, the outcomes are usually very good. Conversely, if a patient's cancer has spread from its organ of origin to distant sites throughout the body, the patient's prognosis is very poor regardless of treatment. The problem is that tumors that are small and confined usually do not cause symptoms. Therefore, to detect these early stage cancers, it is necessary to screen or examine people without symptoms of illness. In such apparently healthy people, cancers are actually quite rare. Therefore it is necessary to screen a large number of people to detect a small number of cancers. As a result, cancer-screening tests are relatively expensive to administer in terms of the number of cancers detected per unit of healthcare expenditure.

A related problem in cancer screening is derived from the reality that no screening test is completely accurate. All tests deliver, at some rate, results that are either falsely positive (indicate that there is cancer when there is no cancer present) or falsely negative (indicate that no cancer is present when there really is a tumor present). Falsely positive cancer screening test results create needless healthcare costs because such results demand that patients receive follow-up examinations, frequently including biopsies, to confirm that a cancer is actually present. For each falsely positive result, the costs of such follow-up examinations are typically many times the costs of the original cancer-screening test. In addition, there are intangible or indirect costs associated with falsely positive screening test results derived from patient discomfort, anxiety and lost productivity. Falsely negative results also have associated costs. Obviously, a falsely negative result puts a patient at higher risk of dying of cancer by delaying treatment. To counter this effect, it might be reasonable to increase the rate at which patients are repeatedly screened for cancer. This, however, would add direct costs of screening and indirect costs from additional falsely positive results. In reality, the decision on whether or not to offer a cancer screening test hinges on a cost-benefit analysis in which the benefits of early detection and treatment are weighed against the costs of administering the screening tests to a largely disease-free population and the associated costs of falsely positive results.

A common strategy to increase the effectiveness and economic efficiency of cancer screening is to stratify individuals' cancer risk and focus the delivery of screening and prevention resources on the high-risk segments of the population. Two such tools to stratify risk for breast cancer are termed the Gail Model and the Claus Model. The Gail model is used as the “Breast Cancer Risk-Assessment Tool” software provided by the National Cancer Institute of the National Institutes of Health on their web site. Neither of these breast cancer models utilizes genetic markers as part of their inputs. Furthermore, while both models are steps in the right direction, neither the Claus nor Gail models have the desired predictive power or discriminatory accuracy to truly optimize the delivery of breast cancer screening or chemopreventative therapies.

These issues and problems could be reduced in scope or even eliminated if it were possible to stratify or differentiate a given individual's risk from cancer more accurately than is now possible. If a precise measure of actual risk could be accurately determined, it would be possible to concentrate cancer screening and chemopreventative efforts in that segment of the population that is at highest risk. With accurate stratification of risk and concentration of effort in the high-risk population, fewer screening tests would be required to detect a greater number of cancers at an earlier and more treatable stage. Fewer screening tests would mean lower test administrative costs and fewer falsely positive results. A greater number of cancers detected would mean a greater net benefit to patients and other concerned parties such as health care providers. Similarly, chemopreventative drugs would have a greater positive impact by focussing the administration of these drugs to a population that receives the greatest net benefit.

One possible way in which to stratify an individual's risk is to consider the individual's genetic traits along with other factors, although conventional techniques in this regard are not altogether satisfactory. Currently, a popular method to identify complex interactions between genetic traits, personal history measures, environmental factors and particular disease states is the case/control associative study. This method examines a group individuals of who have some condition or disease (cases) and an appropriate group of control individuals that do not exhibit this condition or disease. One then looks for some factor that is distributed differently in the group of cases relative to the controls. Classic examples of such studies might be those used to identify the association between cigarette smoking and lung cancer. While most cigarette smokers do not get lung cancer and not all lung cancer victims are cigarette smokers, there is a clear association between cigarette smoking and the risk of developing lung cancer.

One of the reasons for the relative ease in identifying the association between cigarette smoking and lung cancer is that, while clearly more common in lung cancer patients than in the general population, cigarette smoking was a common characteristic of members of general population as well as lung cancer patients. Statistical estimates of the frequency of events in the general population based upon a sample of the general population are more accurate when the events are common. Alternatively, accuracy is more difficult to attain when trying to estimate the frequency of a rare event in the general population based upon a sample. This difficulty in accurately estimating the frequency of rare events in the general population based upon a sample has been known since the 19th century when it was first identified and characterized by the French mathematician, Simeon D. Poisson.

Case/control associative studies compare the frequency of some event or state in the one group (i.e. people with some disease) with the frequency of some event or state in another group (i.e. disease free individuals). For some arbitrary state, assume that the event or state being examined occurs in 50% (frequency=0.5) of the cases and 25% (frequency=0.25) of the controls. Typically, the results of such an analysis is expressed as an Odds Ratio (OR).

Let the frequency of an event or state in the cases be =j.

Let the frequency of an event or state in the controls be =k.

$$OR = \frac{( j / ( 1 - j ) )}{( k / ( 1 - k ) )} = 1.0 / 0.33 = 3.0$$

The event or state being examined is associated with the cases with an OR of 3.0. Because the event or state being examined is fairly common, estimates for j and k are likely to be accurate even if the sample sizes for the case and control populations are fairly modest. Obviously, the accuracy of the assignment of an OR is sensitive to the accuracy of the estimates of the frequencies of the event or state in the case and control populations. Problems arise when the event or state being examined is relatively rare in the cases and/or the controls.

Consider the hypothetical case that in a sample of 500 cases and 500 controls an event or state occurs in 15 cases (j=0.03) and 5 controls (k=0.01). The estimate of the OR would be 3.06. This estimate is very uncertain and likely to be inaccurate because the estimates of j and k are inaccurate. This problem is referred to as the “Poisson Problem”.

Techniques of this disclosure address the Poisson Problem and allow one to effectively stratify or differentiate a given individual's risk from disease (such as cancer) more accurately than is now possible. For these and other reasons that will be apparent to those having ordinary skill in the art, a significant need exists for the techniques described and claimed herein.

### SUMMARY OF THE INVENTION

Particular shortcomings of the prior art are reduced or eliminated by the techniques discussed in this disclosure. In an illustrative embodiment, statistical techniques are used to evaluate large amounts of genetic data to determine if one or more particular genotype combinations are associated with an increased risk for a particular disease. To make such a determination, a multitude of different genotype combinations (easily upwards of 100,000) may be considered to discover evidence of a correlation with the disease.

In one respect, the invention involves a method for statistically identifying an increased risk for disease. A plurality of resampling subsets of a case/control data set for the disease are determined. Disease odds-ratios are determined for different genotype combinations within each resampling subset, thereby generating an odds-ratio distribution. A p-value for each disease odds-ratio within each resampling subset is determined, thereby generating a p-value distribution. An increased risk for disease associated with one or more particular genotype combinations is identified using one or both of the odds-ratio and p-value distributions.

In another respect, the invention involves a method for statistically identifying an increased risk for disease. Disease odds-ratios for different genotype combinations within a case/control data set are determined. Designations for case and control data entries within the data set are randomly permutated to define a plurality of permutated data sets. Permutated odds-ratios for the different genotype combinations are determined for each permutated data set. Empirical p-values for the disease odds-ratios are determined using the permutated odds-ratios, and an increased risk for disease associated with one or more particular genotype combinations is identified using one or both of the disease odds-ratios and empirical p-values.

In another respect, the invention involves computer readable media comprising instructions for carrying out steps mentioned above.

As used herein, “a” and “an” shall not be interpreted as meaning “one” unless the context of the invention necessarily and absolutely requires such interpretation.

As used herein, the phrase “disease” is to be interpreted broadly to encompass any type of disorder.

As used herein, a “genotype combination” refers to a combination of specific alleles of one or more genes. A “genotype combination” encompasses combinations of genetic polymorphisms. By way of example, a one-gene genotype combination for a gene having two alleles A and B may be AA. A different one-gene combination is AB. A two-gene genotype combination may be: a first gene being AA and a second gene being AB. A different two-gene combination may be: the first gene being AB and the second gene being BB, and so on.

Unless otherwise explicitly limited by a claim or by the disclosure itself, generic reference to different “genotype combinations” encompasses different one-gene combinations, two-gene combinations, three-gene combinations, and/or upwards.

As used herein, a “dominance genotype class” is a class of genotypes representing dominance characteristics. For example, a dominance genotype class exhibiting a possible dominance of A over B may be represented as A*, which represents AA or AB. A dominance genotype class exhibiting a possible dominance of B over A may be represented as B*, which represents BB or AB.

As used herein, an odds-ratio “distribution” is a collection of different odds-ratios or a representation of different odds-ratios (e.g., a summary of different odds-ratios or a consolidation of different odds-ratios). A p-value “distribution,” likewise, is a collection of different p-values or a representation of different p-values (e.g., a summary of different p-values or a consolidation of different p-values).

As used herein, an “increased risk” is to be interpreted broadly, as it simply refers to a statistically-significant risk that is higher than that of a general population. In one embodiment, an “increased risk” may be associated with an odds-ratio greater than 1.0.

As used herein, these additional terms shall be interpreted as follows:

“Genome”: All of the DNA an organism inherits from its parent(s). Some viruses have genomes made of RNA instead of DNA, but this is a special case.

“Gene”: Traditionally defined as a complementation group in genetic analysis, in current molecular biology terms, a gene is the total continuous stretch of DNA that is required for the appropriate transcription and post-transcriptional processing of a functional RNA. A gene includes promoter sequences and other cis-acting regulatory sequences, the DNA template for the RNA transcript, and cis-acting sequences required for post-transcriptional processing such as intron splicing and poly-A addition.

“mRNA”: Messenger RNA. A messenger RNA (mRNA) is a functional RNA that directs the synthesis of proteins by ribosomes. This process is called translation. The sequence of amino acids in a protein is determined by the sequence of ribonucleotides in the mRNA as defined by the genetic code. The vast majority of genes in all living organisms, including humans, direct and encode the synthesis of functional RNAs that are mRNAs. There are three parts of a typical mRNA. The front end or 5′ untranslated region (5′ UTR), the open reading frame (ORF) or the portion of the mRNA that is translated into protein, and the back end or 3′ untranslated region (3′UTR). The 5′ UTR and 3′ UTR do not encode parts of the protein, but are important regulatory domains controlling rates of translation and mRNA degradation.

“Allele”: A specific form of a gene. Frequently, the same gene may have a different DNA sequence in different individuals of the same species. These different forms of the same gene are called different alleles of the gene. Basically, all humans have the same set of genes in their genomes. However, we may have dramatically different sets of alleles of these genes. This is why people are different from one another.

“Polymorphism:” In genetic terms, a polymorphism is a site in the genome where different copies of a gene in a population of individuals may have different nucleotide sequences. Various alleles of a gene in a population are typically identical except at the site or sites of polymorphisms. More than one polymorphic site can occur in a single gene. An allele of a gene may be determined by the determination of the genes DNA sequence at the sites at which polymorphisms occur.

“Single Nucleotide Polymorphism (SNP)”: A polymorphism involving a variation at a single nucleotide position in a gene. Some SNPs alter the functions of the proteins encoded by relevant gene. For example, a gene could have two alleles that differ at a single nucleotide position. Such SNPs may also result in a change in the amino acid sequence of a protein and/or a restriction endonuclease recognition site.

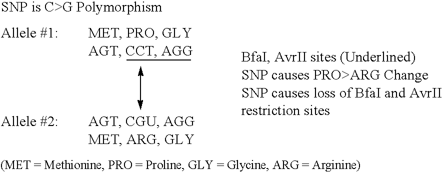

“Genotype”: The specific alleles of one or more genes that an individual possesses in their genome. Since all individuals carry two copies of all autosomal genes, two alleles must be designated for the genotype of all polymorphisms autosomal genes. For the specific example described above, an individual could possess one of the following genotypes, C/C, C/G or G/G.

“Autosomal genes”: Genes encoded on the DNA of the non-sex chromosomes.

“Allelic Frequency”: The proportion of all copies of a gene in a population that are a specific allele. In the example given above, 70% of the copies of the gene in the population could be the C allele and 30% of the copies of the gene in the population could be the G allele. The allelic frequencies for the C and G alleles would be 0.7 and 0.3 respectively. Note that the sum of the allelic frequencies equals 1.0.

“Homozygous”: The state of having a genotype with two copies of the same allele of a polymorphic gene. C/C or G/G in the example given above.

“Heterozygous”: The state of having a genotype with two different alleles of the same polymorphic gene. C/G in the example given above.

“Hardy-Weinberg Equilibrium”: A mathematical model that predicts the genotype frequencies of one or more polymorphic genes in a randomly mating population. In the simplest case, where a single gene is polymorphic at a single site with two alleles that have allelic frequencies of p and q respectively:
(p+q)²=1
or p²+2pq+q²=1

In the example given above, the expected genotype frequency of individuals with the genotype of C/C would be (0.7)²=0.49. One would expect that 49% of individuals in a population would have the genotype of C/C. Similarly, the expected genotype frequencies would be 0.42 (=2×0.7×0.3) for individuals who had the heterozygous genotype C/G. Also, one would expect 0.09 (0.3)² to be the genotype frequency of individuals with the homozygous genotype, G/G.

One can expand this model to predict the genotype frequencies for more than one polymorphic unlinked gene. Consider a second polymorphic gene with two alleles that have the frequencies of r and s respectively. The expected frequencies of genotypes for this second gene would be:
(r+s)²=1
or r²+2rs+s²=1

The expected genotype frequencies for the two genes in combination would be:
(p+q)²×(r+s)²=1

This model can be expanded to predict the genotype frequencies of any number of genes in combination, as will be discussed below.

Other features and associated advantages will become apparent with reference to the following detailed description of specific embodiments in connection with the accompanying drawings.

### BRIEF DESCRIPTION OF THE DRAWINGS

The techniques of this disclosure may be better understood by reference to one or more of these drawings in combination with the detailed description of illustrative embodiments presented herein.

FIG. 1 is a flowchart showing a resampling method for statistically identifying an increased risk for disease, according to embodiments of the present disclosure.

FIG. 2 is a flowchart showing a randomization method for statistically identifying an increased risk for disease, according to embodiments of the present disclosure.

FIG. 3 is a flowchart illustrating the use of Hardy-Weinberg modeling of the controls, according to embodiments of the present disclosure.

### DESCRIPTION OF ILLUSTRATIVE EMBODIMENTS

Bioinformatic techniques of the present disclosure address several shortcomings existing in the prior art. In a representative embodiment, a case/control data set is obtained for one or more diseases. The “case” entries within the data set correspond to patients with a particular disease or condition, and the “control” entries correspond to patients without that disease or condition. The case/control data set includes not only information about whether the patient has or does not have a particular disease or condition, but also genetic information from that patient. For instance, the case/control data may include the genotypes of one or more genes. In a representative embodiment, genotypes of 20 different genes may be included in the case/control data set. In other embodiments, the case/control data set may include other “exposure” factors other than genetic information; for instance, different environmental (e.g., living in proximity to power lines, nuclear plants, toxic waste dumps), lifestyle (e.g., smoker, drug user, lack of exercise), diet (e.g., high-fat, low-carbohydrate), and other factors may be included so that a correlation may be made to determine if certain combinations give rise to an increased risk for disease.

It is one aim of this disclosure to provide techniques allowing one to correlate the presence of a disease with one or more particular genotype combinations of one or more different genes. In lay terms, by analyzing a multitude of genotype combinations, one may uncover a statistical “link” between carrying a particular genotype combination and developing a particular disease. Thus, one may statistically identify an increased risk for disease by simply obtaining genetic information for a patient and determining whether that patient has one or more suspect genotype combinations. Such a patient may be provided an actual quantitative risk value (e.g., “you have a 60% chance of eventually developing breast cancer”) and/or advised that certain preventative measures should be taken. That patient may be more actively monitored and tested to ensure that early detection and treatment may be achieved.

The consideration of all possible genotype combinations (or a large subset) is important given the following assumptions: (1) the risk of a particular disease often only appears with combinations of genes, which is backed-up by observations of smaller risk attributable to the genes when considered one or even two at a time, and (2) particular harmful genotype combinations may often be at least initially un-apparent since they involve what may first appear to be “safe” alleles. Accordingly, there is no way to arrive at suspect combinations through traditional step-wise schemes.

The current teaching in statistics, and particularly in epidemiology, dictates that looking at all possible combinations (or a large subset) of risk factors (often described as a “fishing expedition”) is to be avoided at all costs, primarily because of false-positive issues. Therefore, analysts, perhaps by their upbringing, avoid such an approach. Additionally, there is also the programming requisite of performing a computer-driven analysis of all, or a large subset, of combinations and the challenge of having sufficient computing power and time to run the analysis—not to mention sufficient disk space to store the results.

One main tool for analyzing genetic information within a case/control data set is the odds-ratio (OR) statistic, which approximates relative risk, i.e., the increased risk for developing the disease (e.g., breast cancer) among people in the “exposed” group (the group having a particular combination of factors) compared to those who are not in the exposed group (or compared to the average risk in the general population). Those having ordinary skill in the art will recognize, however, that other statistical tests may now, or in the future, exist for determining relative risk.

Determining which combination(s) correlates to the presence of a particular disease involves analyzing a multitude of different genotype combinations. Consider, for example, a case in which a practitioner is considering genes having only two alleles—A and B. With consideration of dominance, this leads to five genotype classes per gene. The five genotype classes are:
(1) AA;
(2) AB;
(3) BB;
(4) A* (the dominance genotype class for AA, AB); and
(5) B* (the dominance genotype class for BB, AB).

For a combination of two genes there are then 5×5=25 genotype combinations to consider. For a combination of three genes there are then 5×5×5=125 genotype combinations. If one is selecting three genes at a time from a set of 20, there are (20×19×18)/(3×2×1)=1140 different three-gene selections. Each individual selection has three genes and thus has 5×5×5=125 genotype combinations. Therefore, there is a total of 1140×125=142,500 genotype combinations to be considered when selecting three genes at a time from a set of 20.

In one embodiment, an aim is to find genotype combinations that lead to a statistically significantly increased risk for breast cancer. Typically, statistical tests look for a 5% (1 in 20) level of significance. If there were no significantly increased risk and the experiment were repeated a hundred times, then, on average, five of the experiments would give a falsely-positive result. A consequence is that if you were to consider 142,500 experiments (the number of three-gene genotype combinations when three genes are selected at a time from 20 total genes), then, on average, one would have 7,125 false positive results—a number too large to be ignored, especially considering that each of these false positives may frighten or significantly change the lifestyle of a patient.

The problem of a great number of false-positives in the face of testing a multitude of different combinations may be alleviated by considering more conservative levels of significance such 1 in 100 (1425 false positives), 1 in 1000 (142.5 false positives), and so on. However, there is an associated loss of statistical power that leads to increased chance of missing a real result (a falsely negative result).

To circumvent these problems as well as problems in the prior-art, one may utilize one or more aspects of different embodiments of this disclosure—(1) a genotype combination resampling scheme, (2) a genotype combination randomization scheme, and/or (3) a Hardy-Weinberg modeling scheme in combination with the other embodiments. In the resampling scheme, one repeats an experiment over and over (resampling). One randomly selects a subset of cases and controls, calculates test statistics, and then repeats the procedure (e.g., 1000 or more times, limited only by computing power and the patience of the practitioner) to generate a distribution of the odds-ratios. If in 1000 experiments, the observed minimum odds-ratio is greater than 1.0, then this is unlikely to be a false-positive result. This, by itself, however, does not offer a p-value to judge significance. One can, however, calculate asymptotic p-values for each experiment and, hence, generate a distribution of p-values. One may then offer the average p-value as “the” p-value for the experiment.

In the randomization scheme, one may use all available cases and controls from a case/control data set to calculate odds-ratios. Then, one may randomize the designation of case and control (to essentially give the null hypothesis situation), calculate the odds-ratio for the randomized case-control study, and repeat (e.g., 10,000 or more times, limited only by computational power and the patience of the practitioner) to generate the null distribution for the odds-ratios. This distribution may then be used to estimate an empirical p-value for original observed odds-ratios. This technique avoids situations where small counts for a particular combination in either the cases or the controls lead to doubt about the validity of the asymptotic theory used in the resampling scheme.

In the Hardy-Weinberg scheme, one may take advantage of Hardy-Weinberg modeling to, for example, derive a more relevant odds ratio.

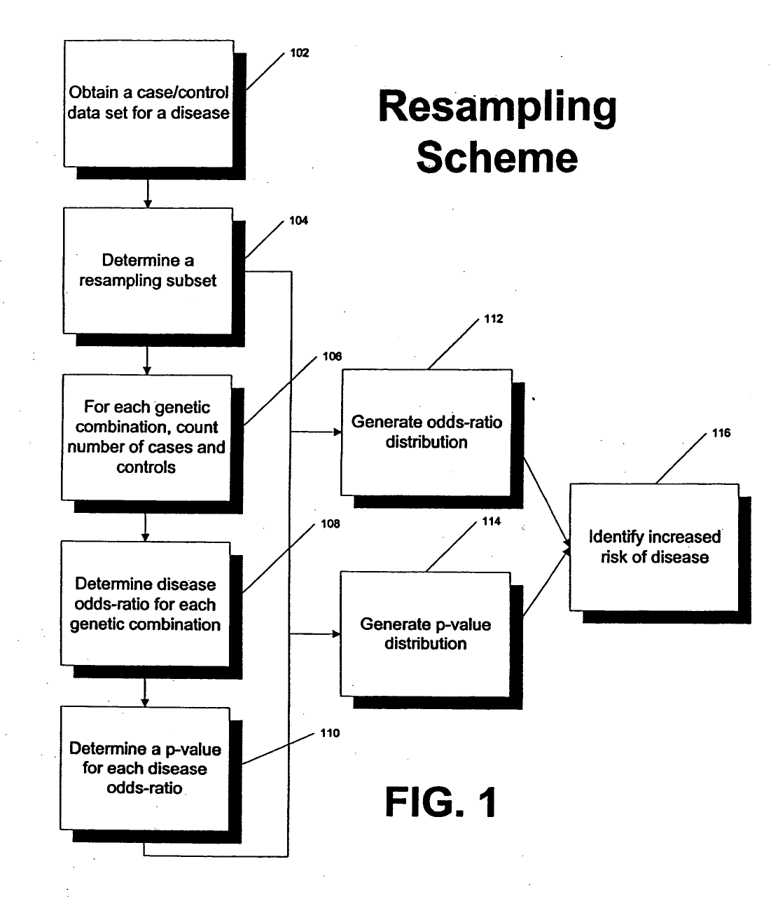

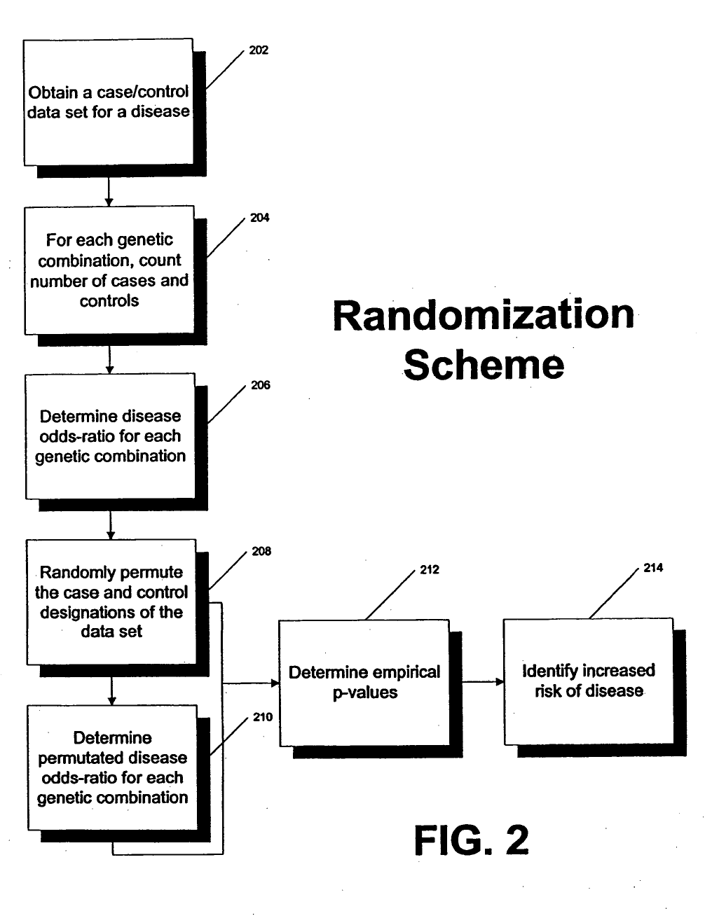

FIGS. 1 and 2 respectively illustrate an exemplary resampling scheme and randomization scheme, each of which is discussed in turn.

FIG. 1 is a flowchart illustrating a resampling method for statistically identifying an increased risk for disease, according to embodiments of the present disclosure. The flowchart includes eight overall steps, although it will be apparent to those having ordinary skill in the art that the number may be smaller through consolidation or greater through additional complementary steps.

In step 102, one obtains a case/control data set. The case/control data set generally includes genetic information from several patients, some of which have a disease (the “case” entries) and some of which do not have the disease (the “control” entries). The size and format of the data set may vary widely according to what application(s) generated the data. In one embodiment, however, the case/control data set may include the following fields, arranged in an array: i.d. #, race, status, disease, age, gene 1, gene 2, gene 3, . . . gene n. The i.d. field may be used to identify a particular patient (by number or a textual identifier). The race field identifies the race of that patient. The status field may be a general field that can be used during processing as a flag or the like. The disease field identifies whether the patient has or does not have a particular disease (hence, it identifies the patient as a case or a control). The age field identifies the age of the patient. Each gene field (labeled 1 through n) includes a genotype for that gene. All of these fields may be filled with numbers only, text and numbers, or any other machine-readable identifier. An appropriate “look-up table” may be used to correlate the identifier with the value or significance of the field.

As will be understood by those having ordinary skill in the art, more or fewer fields may be utilized according to the needs of a particular analysis. In fact, in one embodiment, one may initially analyze the case/control data and eliminate one or more unneeded data entries (samples). For example, one may analyze the case/control data and eliminate all un-genotyped samples—samples for which there is insufficient genetic data. Likewise, samples with a missing age, i.d. #, or any other field may be “weeded-out” from the data set prior to running an analysis.

In step 104, one determines a resampling subset from the case/control data set. A subset of the samples from the case/control data set are selected, or tagged, for processing. In one embodiment, the exact resampling subset may be chosen randomly. In particular, each data entry may be subjected to a random-number test. If a random number is above or below a certain cut-off, the data entry is tagged as falling within the resampling subset. In one embodiment, the “status” field of the case/control data set may be used to tag the entry (e.g., if the entry is selected as being within the resampling subset via the random number test, a “2” may be entered in the field, and if the entry is not selected, a “1” may be entered). In such a randomized selection process, the exact size of different resampling subsets will vary. By changing the nature of the random number test, however, a size distribution may be achieved. For example, if the random number test consists of comparing a random number from 0 to 1 with a threshold of 0.5, it can be assumed that the resampling subset may be about one-half the size of the case/control data set. If a threshold were set at 0.25, the resampling subset may be about three-fourths or one-fourth of the case/control data set, depending on whether the threshold defines inclusion or exclusion from the subset. In other embodiments, one may select resampling subsets using a more fixed routine (as opposed to the randomized method), which, for example, may select a particular number of samples to form a resampling subset.

In step 106, one counts the number of cases and controls (the number of entries having the disease and not having the disease) for each genotype combination within the resampling subset. In one embodiment, the counting is done is follows: count all one-gene genotype combinations, count all two-gene genotype combinations, count all three-gene genotype combinations, etc. Specifically, a first pass of processing (one-gene genotype combinations) may count how many cases and controls exist when gene 1 is AA; how many cases and controls exist when gene 1 is AB; how many cases and controls exist when gene 1 is BB; how many cases and controls exist when gene 2 is AA; . . . ; how many cases and controls exist when gene n is BB (i.e. covering every one-gene genotype combination). A second pass of processing (two-gene genotype combinations) may count how many cases and controls exist when gene 1 is AA and gene 2 is AA; how many cases and controls exist when gene 1 is AB and gene 2 is AA; how many cases and controls exist when gene 1 is BB and gene 2 is AA; . . . etc. (covering every two-gene genotype combination). A third pass of processing (three-gene genotype combinations) may count how many cases and controls exist when gene 1 is AA, gene 2 is AA, and gene 3 is AA; how many cases and controls exist when gene 1 is AA; gene 2 is AA; and gene 3 is AB; etc. (covering every three-gene genotype combination).

In one embodiment, dominance genotype classes are also considered in the counting process. For example, a dominance genotype class exhibiting a possible dominance of A over B may be represented as A*, which represents AA or AB. A dominance genotype class exhibiting a possible dominance of B over A may be represented as B*, which represents BB or AB. Thus, for one-gene genotype combination counting, one may consider how many cases and controls exist when gene 1 is A* and gene 2 is BB; how many cases and controls exist when gene 1 is B* and gene 2 is A*, etc.

Accordingly, in the context of a two allele example utilizing dominance genotype classes and 20 genes in a resampling subset, the one-gene counting of step 106 would involve selecting one gene from the 20. This involves 20 selections. Each selection entails 5 combinations. Therefore 20×5=100 genotype combinations are considered within the resampling subset. The two-gene counting of step 106 would involve selecting a set of 2 genes from the 20. This involves (20×19)/(2×1)=190 selections. Each selection entails 5×5=25 combinations. Therefore 190×25=4750 genotype combinations are considered within the resampling subset. The three-gene counting of step 106 would involve selecting a set of 3 genes from the 20. This involves (20×19×18)/(3×2×1)=1140 selections. Each selection entails 5×5×5=125 combinations. Therefore 1140×125=142,500 genotype combinations are considered within the resampling subset. Combining the number of one-gene, two-gene, and three-gene genotype combinations yields 100+4750+142,500=147,350 combinations being considered within the resampling subset. As will be apparent, considering 4 gene combinations, five-gene combinations, and so on, entails the consideration of a far greater number of combinations, although the methodology is the same. Likewise, selecting from a larger group of genes than 20 would entail more counting. Likewise, the larger the resampling group, the more combinations will need to be considered (but will be significantly lower than if every data entry in the entire case/control data set were used).

With the benefit of the present disclosure, those having ordinary skill in the art will recognize that the size of the case/control data set, the resampling subset, and the extent of combinations (i.e., one-gene vs. two-gene, vs. three-gene, vs. n-gene) simply depends upon the computing power available to the practitioner. As computing resources continue to improve and become more inexpensive, it is anticipated that practitioners may routinely consider 5, 6, 7, 8, 9, 10, 11, 12, etc. gene-combinations from a set of 20, 30, 40, 50, etc. genes from larger and larger overall case/control data sets. These numbers are exemplary only, and not limiting. Any number may be selected using techniques disclosed herein, or their equivalents.

In step 108, one determines a disease odds-ratio for each genotype combination within the resampling subset. In one embodiment, this may be done using 2×2 matrices:

| | cases | controls |
|---|---|---|
| with genotype combination | a | b |
| without genotype combination | c | d |

where the odds-ratio would then be: (a×d)/(b×c). In the example given above in which 1, 2, and 3-gene combinations are counted from a group of 20 genes, there would be 147,350 odds-ratios calculated.

In step 110, one determines a p-value for each disease odds-ratio. The calculation of the p-value may be done by any of the several methods known in the art. In one embodiment, the p-value may be calculated using the following formulae:
y=ln((a×d)/(b×c));
V=1/a+1/b+1/c+1/d; and
u=(y×y)/V
the p-value, p=Prob(X>u), the probability that X is greater than u, where X is distributed as a chi-squared variable with one degree of freedom.

Following step 110, the process loops back to step 104, as illustrated by the looping arrow in FIG. 1. This signifies that once the odds-ratio and p-values are determined within a resampling subset, a new resampling subset is then chosen, and steps 106, 108, and 110 are repeated. In other words, a new resampling subset is selected, the number of cases and controls are counted for each genotype combination, odds-ratios are calculated for each combination, and p-values are calculated for each odds-ratio.

The number of times this loop continues is up to the practitioner and depends on the number of resampling runs that are needed or desired. In one embodiment, the loop continues about 1000 times, although any number suitable to generate statistically significant results may be chosen. If the randomized resampling selection method is used (as described above), the exact size of each resampling group may vary.

Calculating odds-ratios and p-values for several resampling subsets leads to the generation of an odds-ratio distribution and p-value distribution. This is shown as steps 112 and 114 respectively in FIG. 1. For example, consider the first “run” of the flowchart of FIG. 1—it may lead to the calculation of, e.g., 147,350 odds-ratios and 147,350 corresponding p-values. When a second resampling subset is chosen, another 147,350 odds-ratios and 147,350 p-values are generated. When a third resampling subset is chosen, another 147,350 odds-ratios and 147,350 p-values are generated, and so on. Suppose that this is repeated 1,000 times, thus generating 1,000 sets of 147,350 odds-ratios and 147,350 p-values.

Keeping track of the odds-ratios and p-values may be done in any number of ways suitable for managing large amounts of data. In one embodiment, the odds-ratios and p-values for particular genotype combinations may be consolidated into averages, means, or the like. Standard deviations may be calculated, or any other statistical signifier as needed. Odds-ratios and/or p-values falling above or below certain cutoffs may be disregarded or deleted. The data may be grouped according to need into one or more summary reports, spreadsheets, or the like to efficiently distill the information into a more readable, useful form.

In one embodiment, the data within the distributions may be sorted to identify different genotype combinations leading to particular average odds-ratios and/or average p-values. In one embodiment, the genotype combinations giving the highest average odds-ratios may be selected from the distribution and their corresponding average p-value may be presented as “the” p-value for that combination. As one of ordinary skill in the art will appreciate, once the odds-ratio and p-value distributions are generated in steps 112 and 114, practitioners may interpret the results and present and/or summarize those results in numerous ways other than averaging and sorting.

In general, the distributions allow the practitioner to identify an increased risk of the disease being considered in the resampling subsets, as illustrated in step 116 of FIG. 1. In one embodiment, a numerical risk factor may be assigned based upon one or both of the odds-ratio and p-value distributions. For instance, given a particular average odds-ratio for a particular genotype combination existing in the patient, a practitioner may be able to advise that the patient has, e.g., a heightened chance of developing breast cancer. If a look-up table is created correlating average odds-ratios (and, optionally, p-values) to numerical probabilities, one may be able to advise that the patient has, e.g., a 60% chance of developing breast cancer. In either scenario, the patient may be able to engage in more preventative measures, and she may be able to schedule more frequent doctor appointments so that the disease, if it does develop, can be detected early.

The resampling scheme of FIG. 1 effectively allows the practitioner to generate statistically significant data while reducing the impact of errors, since the results are ultimately averaged or otherwise distilled from several different resampling experiments. In other words, rather than analyzing each genotype combination from the entire case/control data set once, the combinations can be analyzed as many times as desired (e.g., thousands of times) in the form of smaller, resampling subsets.

In a generalized embodiment of the methods of FIG. 1, one may use a different statistical test other than the odds-ratio for each genotype combination. In fact, any statistical test may be utilized. Likewise, other signifiers of significance besides p-values may be optionally used. Further, in addition (or alternative to) considering different genotype combinations, one may also consider different combinations of environmental factors, diet factors, or any other measurable “exposure” phenomenon to discover a link or correlation between a certain characteristic and the development of a disease.

FIG. 2 is a flowchart illustrating a randomization method for statistically identifying an increased risk for disease, according to embodiments of the present disclosure. The flowchart includes seven overall steps, although it will be apparent to those having ordinary skill in the art that the number may be smaller through consolidation or greater through additional complementary steps.

In step 202, one obtains a case/control data set. The description of step 102 of FIG. 1 applies to this step, so it will not be repeated.

In step 204, one counts the number of cases and controls (the number of entries having the disease and not having the disease) for each genotype combination within the entire case/control data set (as opposed to a resampling subset as done in FIG. 1). Of course, however, samples may be weeded-out of the case/control data set as is the case in the resampling scheme. As also was the case with the methodology of FIG. 1, one may count one-gene combinations first, two-gene combinations second, three-gene combinations third, and so on. Further, dominance genotype classes may be considered in the counting process.

Accordingly, a two allele example utilizing dominance genotype classes and 20 genes in case/control data set would involve the consideration of 147,350 genotype combinations.

In step 206, one determines a disease odds-ratio for each genotype combination within the case/control data set. In one embodiment, this may be done using 2×2 matrices:

| | cases | controls |
|---|---|---|
| with genotype combination | a | b |
| without genotype combination | c | d |

where the odds-ratio would then be: (a×d)/(b×c).

Having calculated (the observed) odds ratios for the genotype combinations within the case/control data set a single time (as opposed to calculating odds-ratios for each of several resampling subsets), one then proceeds to step 208. In step 208, one randomly permutes designations for case and control data entries within the data set to define a permutated case/control data set. For example, consider a data entry that has a field signifying whether the patient has a disease—the field has a value of 2 if the disease is present (a “case” entry) and a value of 1 if the patient does not have the disease (a “control” entry). Step 208 randomly switches the disease field from 1 to 2 or vice versa. For example, for each data entry, the disease field may be subjected to a randomized test to determine if the field's entry should be a 1 or a 2. For instance, a random number may be compared to a threshold. If the random number exceeds the threshold, the value will be a 1. A permutated case/control data set is accordingly defined.

In one embodiment, the total number of cases and controls is kept constant despite the random permutations. This may be done in any number of suitable ways. In one embodiment, once the number of cases or controls in the permutated data set reaches the number of cases or controls in the original case/control data set, the random permutations end.

Step 210 of FIG. 2 is similar to step 206, except that in step 210, the odds ratios being calculated are for the permutated data set, not the original case/control data set.

Following step 210, the process loops back to step 208, as illustrated by the looping arrow in FIG. 2. This signifies that once the odds-ratio are determined for a permutated data set, a new permutated data set subset is then chosen, and step 210 is repeated. In other words, a new permutated data set is generated, the number of cases and controls are counted for each genotype combination, and odds-ratios are calculated for each combination.

The number of times this loop continues is up to the practitioner and depends on the number of randomization runs is desired. In one embodiment, the loop continues about 10,000 times, although any number suitable to generate statistically significant results may be chosen.

The randomization of case and control essentially provides the null-hypothesis situation. Calculating the odds-ratio for the randomized case/control study generates the null distribution for the odds-ratios, which can then be used to estimate empirical p-values for each of the original odds-ratios calculated in step 206 of FIG. 2. The calculation of empirical p-values is illustrated as step 212. One suitable way of calculating empirical p-values is as follows:

Arrange the “n” number of odds-ratios for a particular combination from the randomization procedure in order of increasing value. Let G be the number of these odds-ratios that equal or exceed the observed odds-ratio for the combination. Then, the empirical p-value, p=G/n. For n=10,000, the p-value would therefore be G/10,000.

As with the embodiment of FIG. 1, the different odds-ratios and p-values may be sorted to identify different genotype combinations within a range of odds-ratios and/or empirical p-values. In one embodiment, the genotype combinations giving the highest odds-ratios may be selected and their corresponding empirical p-value may be presented as “the” p-value for that combination. As one of ordinary skill in the art will appreciate, once the odds-ratios and p-values are generated, practitioners may interpret the results and present and/or summarize those results in numerous ways.

In step 214, one uses one or both of the odds ratios of step 206 and the p-values of step 212 to identify an increased risk of the disease being considered in the case/control data set. In one embodiment, a numerical risk factor may be assigned based upon one or both of the odds-ratio and empirical p-value, as explained in the context of FIG. 1.

The randomization scheme of FIG. 2, through its calculation of empirical p-values, advantageously avoids situations where small counts for a particular genotype combination in either the cases or controls in the original case/control data set lead to doubt about the validity of the asymptotic theory (for calculating p-values, as done in FIG. 1).

In a generalized embodiment of the methods of FIG. 2, one may use a different statistical test other than the odds-ratio for each genotype combination. In fact, any statistical test may be utilized. Likewise, other signifiers of significance besides p-values may be optionally used. Further, in addition (or alternative to) considering different genotype combinations, one may also consider different combinations of environmental factors, diet factors, or any other measurable “exposure” phenomenon to discover a link or correlation between a certain characteristic and the development of a disease.

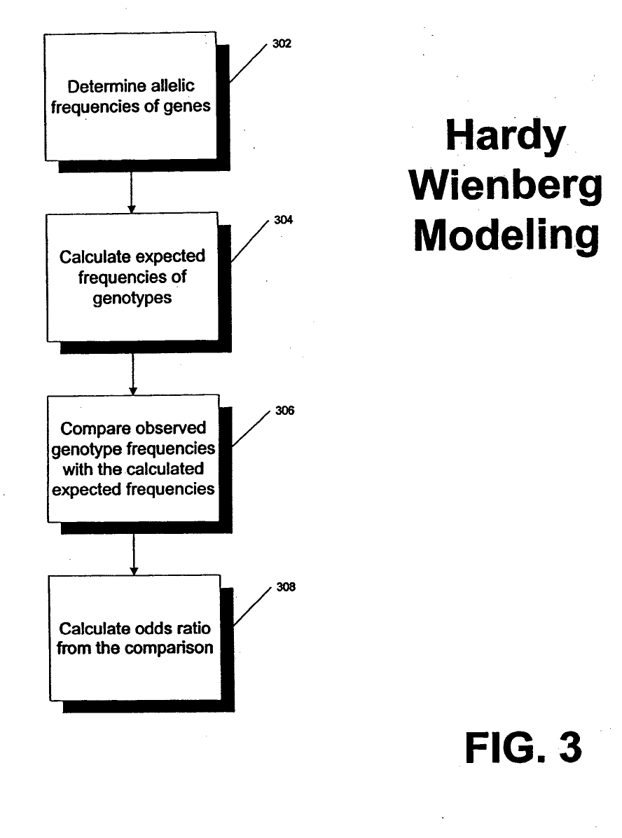

FIG. 3 is a flowchart illustrating the use of Hardy Weinberg modeling to derive a more relevant odds ratio, which may be used with either the techniques of FIG. 1 or FIG. 2 (or a combination of FIGS. 1 and 2). It will be apparent to those having ordinary skill in the art that the number of illustrated steps may be smaller through consolidation or greater through additional complementary steps.

Before explaining the individual steps of FIG. 3, it is useful to explain, in general, Hardy Weinberg modeling (a brief explanation is given in the Summary section, above). If one has knowledge of the allelic frequencies of individual alleles, Hardy-Weinberg Equilibrium models predict the frequency of any genotype for any combination of alleles for any number of unlinked genes in a population. Consider the hypothetical example of three genes (genes 1, 2 and 3). Each gene has two alleles with known allelic frequencies: p and q for gene 1; r and s for gene 2; and t and u for gene 3. The distribution of genotypes for these three genes in the population is:
(p+q)²x(r+s)²×(t+u)²=1
Expanded as:
t²r²p²+2pqt²r²+t²r²q²+2rst²p²+4rspqt²+2rst²q²+t²s²p²+2pqt²s²+t²s²q²+2tur²p²+
4tupqr²+2tur²q²+4tursp²+8turspq+4tursq²+2tus²p²+4tuqs²+2tus²q²+u²r²p²+2pqu²r²+u²r²q²+2rsu²p²+4rspqu²+2rsu²q²+u²s²p²+2pqu²s²+u²s²q²=1

There are 27 possible genotypes. For simplicity, assume the allelic frequencies of q, s, and u are each 0.35. (Allelic frequencies of p, r, and t all equal 0.65). Consider the frequency of individuals with the genotype of gene 1=p/q, gene 2=s/s, and gene 3=u/u. One may write this complex genotype as p/q, s/s, u/u. The frequency of this genotype as predicted by Hardy-Weinberg Equilibrium will be 2pqu²s². This is equal to (2×0.65×0.35)×(0.35²)×(0.35²) or 0.020. Even though all of these alleles are common in the population, the complex genotype is fairly rare. The Poisson Problem makes it very difficult to accurately estimate the frequency of such a rare event from a sample of the population.

Alternatively, it is possible to accurately estimate the frequency of an event that occurs with a frequency of 0.35 even with a modest sample size. Since the frequency of the rare event can be predicted from knowledge of the frequencies of the common events, the predicted frequencies of the rare events are more accurate than the observed frequencies from a sample for estimating the actual frequencies of the rare events in the population from which the sample was obtained. By only observing common events, the entire Poisson Problem is avoided in the controls.

Operationally, data from the controls may be analyzed to determine the allelic frequencies of the genes being examined. The allelic frequencies can be used to calculate the expected frequencies of complex genotypes. Then, the observed frequencies of the complex genotypes in the cases can be compared to the calculated genotypes from the controls to derive the relevant odds ratios. This method removes the Poisson Problem from the denominator of the odds ratio calculation (k), and thus makes the determination of the odds ratio more accurate.

These steps are illustrated in FIG. 3. In step 302, one determines allelic frequencies of genes. In terms of the example above, this would amount to the determination of p, q, r, s, t, and u by analyzing a data set. In step 304, one calculates expected frequencies of one or more genotypes. This step utilizes the Hardy Weinberg equation, discussed above. In step 306, genotype frequencies observed from direct observation of a data set are compared with those calculated in step 304. Through this comparison, one may readily derive an odds ratio, which removes or reduces the Poisson Problem, in step 308.

There are at least two general embodiments of the application of Hardy-Weinberg modeled genotype frequencies for controls in the context of this disclosure. In the first, the allelic frequencies for the individual examined genes are determined. The expected genotype frequencies for all one, two, three, four or more (as desired) combinations of genes are then calculated using the Hardy-Weinberg model. These expected genotype frequencies are then compared to the observed frequencies of the same genotypes in the cases in each round of resampling. Odds Ratios, p-values and other statistics as are desired are calculated as described before except that the Hardy-Weinberg modeled genotype frequencies are substituted for observed genotype frequencies in the controls.

In a second embodiment, resampling of cases and controls is performed as described before. The allelic frequencies of all polymorphisms are then determined for the resampled dataset for the controls. Hardy-Weinberg modeling is then used to determine the predicted genotype frequencies for the one, two, three or more (as desired) combinations of genes in the controls for the resampled data. The predicted genotype frequencies are then used in comparisons with the observed genotype frequencies in the resampled cases. Odds ratios, p-values and other desired statistics are calculated as described before except that the Hardy-Weinberg modeled genotype frequencies are substituted for observed genotype frequencies in the controls. In this embodiment, the Hard-Weinberg modeling is repeated with each round of resampling.

An essence of the Hardy-Weinberg modeled predictions of genotype frequencies is that they are a more accurate estimate of the true frequencies of relatively rare genotypes in a large population than can be observed from a sample.

The following examples are included to demonstrate specific, non-limiting embodiments of this disclosure. It should be appreciated by those of skill in the art that the techniques disclosed in the examples that follow represent techniques discovered to function well in the practice of the invention, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Techniques of this disclosure provide data analysis strategies to identify combinations of genetic polymorphisms and personal history measures that are associated with varying degrees of risk for developing breast cancer. These strategies are broadly applicable to many similar problems involving the interactions of many genes and many environmental factors in determining risk of developing complex diseases. Risk of developing other types of cancer, heart disease and diabetes may be considered. Additionally, one may use the techniques to predict the efficacies of various medical treatments. In short, these are methods to quantitatively dissect the complex, multifactoral interactions between genes and environmental factors to predict outcomes in medical or biological systems.

At least three main embodiments typify this disclosure:

1. Resampling of data.

2. Generating a null hypothesis for genetic association by randomly assigning data from cases and controls into sets of pseudo-cases and pseudo-controls.

3. Using calculated Hardy-Weinberg equilibrium estimates of the frequencies of complex genotypes to model an infinitely large population of controls.

As mentioned before, one may identify associations between complex genotypes involving alleles for many different genes in combination and evaluate the risk of being diagnosed with breast cancer. One may also examine interactions between complex genotypes and certain personal history and environmental factors to evaluate their aggregate association with the risk of developing breast cancer. A significant problem with currently used statistical techniques is that this type of multivariate (multi-gene/allele) analysis divides the population into many small groups. In an exemplary analysis, the populations of cases and controls may be divided into groups that each occur at a frequency on the order of 1% (j and k ˜0.01). In this range, estimates of occurrence frequencies and therefore odds ratios may be inaccurate.

To overcome these inaccuracies, traditional study design requires inordinately large sample sizes. The techniques of this disclosure include a set of novel, powerful statistical methods that permit accurate estimates of odds ratios with, while still large, relatively smaller sample sizes. While one may focus on estimating risk of developing breast cancer, the analytical methods described herein are immediately applicable to a wide variety of other problems in which multivariate genetic analysis subdivides the population into many small groups.

Statistical Methods—Limiting the Impact of the Poisson Problem:

Resampling

As described by Poisson, there is very high variability in the number of rare events that are observed in any sample of a large population. Operationally, this means that in a series of samples from a population, a disproportionate number of samples will contain a significant overrepresentation of the rare event while other samples will contain too few or no events. As the frequency of rare events in the cases and controls become small, the estimate of the odds ratio approaches j/k. If the these estimates of j and k become highly variable from one sample to the next, then the estimate of the relevant odds ratio becomes highly variable. The scientific literature is replete with examples of multiple independent case/control studies that observed widely different and sometimes contradictory odds ratios for the associations of relatively rare events with a particular disease state.

A solution to this problem explained in this disclosure is to reduce the variance in the estimate of the odds ratio by resampling data to create a population of odds ratio estimates that has a smaller variance than can be obtained by a single observation of the same data.

Operationally, one may begin with a sample set large enough to observe multiple examples of the rare event in both the cases and controls. Empirically, estimates of the odds ratios become problematic if there are fewer than seven independent observations of the rare event in either the cases or controls. More than seven independent observations in both the cases and controls are preferred. Next one may assume that the distribution of these rare events in the sample is representative of their distribution in the entire population of cases and controls. One may then randomly select cases and controls from the data set until a significant portion of the total number of cases and controls have been resampled in the data. In one embodiment, one may select 50-80% of the total data. One may then calculate the odds ratio and some other statistics (e.g., any statistic known in the art and suitable for further characterizing the data) for this resampled data set. The results may be saved in a separate “resampling results” database. This process may then be repeated many times, in one embodiment about 500 times. One may then go to the resampling database and calculate the mean odds ratio and a variety of other statistics. The odds ratio for the rare event will be the same (or very nearly the same) as was the odds ratio calculated for the entire data set. However, the variance of the odds ratio from the resampled data set will be smaller. Accordingly, the impact of extreme values created by the Poisson Problem has been reduced. Using this methodology, one is actually creating a model of a data set that is larger than the existing data and hypothesizing that modeled data set is more representative of the entire population than any portion of the existing data.

This technique allows one to examine many thousands of combinations of alleles from many genes together with selected personal history measures and environmental factors. Each of these many combinations is represented as a relatively rare event in the populations of cases and controls. For each of these combinations, one may perform the analysis described above using software suitable for carrying out the steps described herein. One suitable example is given in Example 2, below.

Creating a Null Hypothesis

Another technique described above involves creating a null hypothesis that the rare event being examined is not associated with the disease or state being investigated. Any odds ratio that deviates from 1.0 in cases relative to the controls may be simply an artifact caused by the Poisson Problem. If this null hypothesis is true, then the data from the cases is just a resampling of the same population as the controls. So, let one combine all the data from both the cases and controls together in to one big data set. Now, resample this data and randomly assign individuals to the case group or the control group. Since both groups contain randomly assigned assortments of cases and controls, let one call these groups pseudo-cases and pseudo-controls. Next, calculate the odds ratio and other statistics and save these results to a results database. One may repeat this process many times, in one embodiment about 500 times. One can now calculate the mean odds ratio and standard deviation of the odds ratio. The expected result will be that the mean odds ratio will be 1.0. One can use these statistics to determine the probability that the odds ratio from the real data (actual cases and actual controls) is really just a resampling of the data from the null hypothesis.

Hardy-Weinberg Modeling of the Controls

Given that one has knowledge of the allelic frequencies of the individual alleles, Hardy-Weinberg Equilibrium models predict the frequency of any genotype for any combination of alleles for any number of genes in a population. The assumptions are that the population is a random mating pool and that the genes are unlinked (i.e. they are not located near each other in the genome). These assumptions appear to be met for most of the genes being examined by the inventors.

The Hardy-Weinberg model predicts the frequencies of genotypes in a very large if not infinitely large population of controls. The Hardy-Weinberg modeling of the controls can be embedded into either of the two methods described above.

### EXAMPLE 2

The Intergenetics Breast Cancer Cohort is designed as a classic case-control study: ˜1000 cases, ˜4000 controls. The main tool for the analysis is the odds-ratio statistic, which approximates the relative risk, i.e., the increased risk for developing breast cancer among people in the exposed group compared to those who are not (or compared to the average risk in the general population). Exposure in this example is carrying a particular combination of alleles at a set of genes.

The genes being considered typically have two alleles, termed A and B for convenience. With consideration of possible patterns of dominance, this leads to five genotype classes per gene. For a combination of two genes there are then 5×5=25 genotype combinations to consider, 125 for combinations of three genes. Therefore, with a set of twenty genes from which to select three at a time (1140 selections) there are 142,500 three gene combinations to be considered.

A goal of this example is to provide software that may find genotype combinations that lead to a statistically significantly increased risk for breast cancer. The software source code submitted as a computer program listing appendix on CD utilizes a resampling scheme analogous to that of FIG. 1. With the benefit of this disclosure, those having ordinary skill in the art can readily modify the source code to achieve the randomization techniques discussed in FIG. 2 as well. Although the source code is in FORTRAN, any other computer language suitable for carrying out the details of the statistical operations may be used.

The computer program listing appendix on CD is one embodiment of FORTRAN source code for a resampling-scheme program. The program calls the subroutines in the source code given subsequently. Those subroutines calculate odds ratios and theoretical p-values. The final piece of source code is a repetitively-called outputting subroutine.

With the benefit of the present disclosure, those having skill in the art will comprehend that techniques claimed herein and described above are example embodiments only and may be modified and applied to a number of additional, different applications, achieving the same or a similar result. For instance, techniques of FIG. 1 may be used in combination with those of FIG. 2. Specifically, one may calculate empirical p-values in the resampling scheme of FIG. 1, and one may use resampling techniques in the randomization methodology of FIG. 2. Similarly, the techniques of FIG. 3 may be used in conjunction with those of FIG. 1, FIG. 2, or a combination of FIGS. 1 and 2. The claims attached hereto cover all such modifications that fall within the scope and spirit of this disclosure.

1. A method for statistically identifying an increased risk for disease, the method comprising:
determining a plurality of resampling subsets of a case/control data set for the disease;
determining disease odds-ratios for different genotype combinations within each resampling subset, thereby generating an odds-ratio distribution;
determining a p-value for each disease odds-ratio within each resampling subset, thereby generating a p-value distribution; and
identifying an increased risk for disease associated with one or more particular genotype combinations using one or both of the odds-ratio and p-value distributions.

2. The method of claim 1, wherein the disease odds-ratios or the p-values are determined using Hardy-Weinberg modeled predictions of genotype frequencies.

3. The method of claim 1, the plurality of resampling subsets being of different size.

4. The method of claim 3, the size of each resampling subset being determined randomly.

5. The method of claim 1, the different genotype combinations comprising one or more combinations of dominance genotype classes.

6. The method of claim 1, the different genotype combinations arising from the genotype combinations associated with up to three polymorphic sites being selected from a group of many polymorphic sites in many genes.

7. The method of claim 1, wherein identifying an increased risk for disease comprises assigning a numerical risk factor based upon one or both of the odds-ratio and p-value distributions.

8. The method of claim 1, the plurality of resampling subsets comprising between 2 and 1000 resampling subsets.

9. The method of claim 1, the plurality of resampling subsets comprising between 1,000 and 1,000,000 resampling subsets.

10. The method of claim 1, the plurality of resampling subsets comprising between 1,000,000 and 100,000,000 resampling subsets.

11. The method of claim 1, further comprising eliminating one or more un-genotyped samples from the resampling subsets.

12. The method of claim 1, the identifying comprising considering one or both of an average odds-ratio or an average p-value from the odds-ratio and p-value distributions.

13. A method for statistically identifying an increased risk for disease, the method comprising:
determining disease odds-ratios for different genotype combinations within a case/control data set;
randomly permuting designations for case and control data entries within the data set to define a plurality of permutated data sets;
determining permutated odds-ratios for the different genotype combinations for each permutated data set;
determining empirical p-values for the disease odds-ratios using the permutated odds-ratios; and
identifying an increased risk for disease associated with one or more particular genotype combinations using one or both of the disease odds-ratios and empirical p-values.

14. The method of claim 13, the different genotype combinations comprising one or more combinations of dominance genotype classes.

15. The method of claim 13, the different genotype combinations arising from the genotype combinations associated with up to three polymorphic sites being selected from a group of many polymorphic sites in many genes, each polymorphic site having two or more allelic variants.

16. The method of claim 13, wherein identifying an increased risk for disease comprises assigning a numerical risk factor based upon one or both of the one or both of the disease odds-ratios and empirical p-values.

17. The method of claim 13, further comprising eliminating one or more un-genotyped samples from the case/control data set.

18. Computer readable media comprising instructions for:
determining a plurality of resampling subsets of a case/control data set for the disease;
determining disease odds-ratios for different genotype combinations within each resampling subset, thereby generating odds-ratio distributions;
determining a p-value for each disease odds-ratio within each resampling subset, thereby generating p-value distributions; and
identifying an increased risk for disease associated with one or more particular genotype combinations using one or both of the odds-ratio and p-value distributions.

19. The media of claim 18, further comprising instructions for determining the disease odds-ratios or the p-values are using Hardy-Weinberg modeled predictions of genotype frequencies.

20. The media of claim 18, the resampling subsets being of different size.

21. The media of claim 20, the size of each resampling subset being determined randomly.

22. The media of claim 18, the different genotype combinations comprising one or more combinations of dominance genotype classes.

23. Computer readable media comprising instructions for:
determining disease odds-ratios for different genotype combinations within a case/control data set;
randomly permuting designations for case and control data entries within the data set to define a plurality of permutated data sets;
determining permutated odds-ratios for the different genotype combinations for each permutated data set;
determining empirical p-values for the disease odds-ratios using the permutated odds-ratios; and
identifying an increased risk for disease associated with one or more particular genotype combinations using one or both of the disease odds-ratios and empirical p-values.

24. The media of claim 23, the different genotype combinations comprising one or more combinations of dominance genotype classes.
